(19) United States
(12) Patent Application Publication
(10) Pub. No.: US 2007/0093517 A1
(43) Pub. Date: Apr. 26, 2007

(54) Local anesthetic compositions

(76) Inventor: Gary Newton, Fayetteville, NC (US)

Correspondence Address: JENKINS, WILSON, TAYLOR & HUNT, P. A., 3100 TOWER BLVD, SUITE 1200, DURHAM, NC, 27707, US

(21) Appl. No.: 11/491,874

(22) Filed: Jul. 24, 2006

Related U.S. Application Data
(60) Provisional application No. 60/729,621, filed on Oct. 24, 2005.

(51) Int. Cl. A61K 31/485

(52) U.S. Cl. 514/282

(57) ABSTRACT

The treatment of pain and related use by topical administration of an N-substituted-14-hydroxydihydronormorphine to a subject in need thereof. In some embodiments, the N-substituted-14-hydroxydihydronormorphine is nalbuphine.

### RELATED APPLICATIONS

This application claims the benefit of U.S. Provisional Patent Application Ser. No. 60/729,621, filed Oct. 24, 2005; the disclosure of which is incorporated herein by reference in its entirety.

### TECHNICAL FIELD

The presently disclosed subject matter relates generally to the administration, and in particular embodiments, the topical administration, of N-substituted-14-hydroxydihydronormorphines (e.g., nalbuphine) to provide local anesthetic effects in a subject in need of such treatment.

Abbreviations

| | |
|---|---|
| CU | Clinical units |
| BT | body temperature |
| ° C. | degrees Celsius |
| cm | centimeter |
| ED₅₀ | dose that produce a 50% effect |
| FM | formula mass |
| kg | kilogram |
| min | minute |
| mg | milligram |
| ml | milliliter |
| n | number |
| % | percent |

### BACKGROUND ART

An anesthetic is a drug that causes loss of physical sensation in a subject. There are many types of anesthetics commonly in use today, such as general and local anesthetics. A local anesthetic acts via a loss of sensation in a localized area of administration in the body. The mechanism by which local anesthetics induce their effect is generally thought to be based upon their ability to locally interfere with the initiation and transmission of a nerve impulse, e.g., interfering with the initiation and/or propagation of a depolarization wave in a localized area of nerve tissue.

Frequently, local anesthetics are administered to a subject by an injection. However, there are many instances where the administration of a topical anesthetic is desired. One such instance can be where the pain resulting from the injection is undesirable. Another such instance is the administration of injections to young children. Many children fear an injection more than the pain they are suffering. As a result, even the sight of a hypodermic needle (such as in an emergency situation) can create agitation in the patient. Another instance can arise in veterinary practice. The administration of injections to animals can cause them pain and make them more difficult to control.

Further, there are many types of conditions, injuries, wounds, and the like where the use of an applicator no matter how soft and pliable can create pain or increase the risk of further harm. One such example is an eye wound. The sensitive nature of the ocular tissue makes it very difficult for a topical anesthetic to be applied. Those of ordinary skill in the art will recognize that there are numerous other types of instances it would be desirable to administer a topical anesthetic without the use of an applicator that touches the tissue surface. Thus, there is a continuing need in the art for a topical anesthetic without an applicator to touch tissue.

Morphine has been used since the eighteenth century as an anesthetic. However, morphine has a number of severe side effects, including but not limited to a highly addictive nature. Many patients are biased against the use, need, or possession of morphine based on the associated negative side effects.

U.S. Pat. No. 3,393,197 issued to Pachter and Matossian on Jul. 16, 1968 discloses morphine derivatives with fewer side effects, specifically N-substituted-14-hydroxydihydronormorphines of the formula: 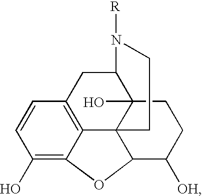
where R can be an aliphatic group.

The presently disclosed subject matter relates generally to methods of treating pain through local topical use, and in some embodiments topical use, of an N-substituted-14-hydroxydihydronormorphine in a subject in need of such treatment.

Further, the presently disclosed subject matter relates in some embodiments to the use of an N-substituted-14-hydroxydihydronormorphine in an eyedrop formulation to provide local topical anesthetic treatment in a subject in need of such treatment.

### SUMMARY

Disclosed are methods of and compositions for treating pain in a subject in need thereof. The methods can comprise topically administering to a site of pain in the subject a therapeutically effective amount of an anesthetic formulation comprising an N-substituted-14-hydroxydihydronormorphine of Formula (I):

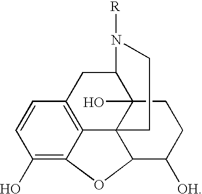

In some embodiments, a method of providing local anesthetic to a site of pain in a subject in need thereof comprising topically administering a therapeutically effective amount of an anesthetic formulation comprising N-substituted-14-hydroxydihydronormorphines of Formula (I):

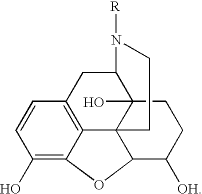

In some embodiments, the R group of Formula (I) is alkyl, substituted alkyl, cycloalkyl, substituted cycloalkyl, aryl, or substituted aryl. In some embodiments, the R group of Formula (I) is selected from the group consisting of allyl, methylallyl, dimethylallyl, propynyl, methylpropynyl, cyclopropylmethyl, cyclobutylmethyl, and propyl.

In some embodiments, the compound is nalbuphine.

In some embodiments, the anesthetic formulation is topically administered in the eye of the subject.

In some embodiments, the anesthetic formulation comprises an N-substituted-14-hydroxydihydronormorphine at a concentration of about 0.1% to about 5% by weight, optionally at about 1% by weight.

In some embodiments, the anesthetic formulation comprises a pharmaceutically acceptable carrier suitable for topical application. In some embodiments, the anesthetic formulation comprises a pH-adjusting agent.

In some embodiments, the anesthetic formulation is adjusted to a pH range of about 6.5 to about 7.5.

In some embodiments, the anesthetic formulation comprises one or more pharmaceutically acceptable additives selected from the group consisting of a carrier, a lubricant, an emulsifying agent, a wetting agent, a bodying agent, a thickener, a tonicity agent, a comfort-enhancing agent, a solubilizing aid, an antioxidant, a stabilizing agent, and combinations thereof.

In some embodiments, the anesthetic formulation comprises one or more opthalmologically acceptable additives selected from the group consisting of a preservative, a surfactant, a viscosity enhancer, a penetration enhancer, a buffer, water and combinations thereof.

In some embodiments, the carrier comprises one or more of water, water-miscible solvents, phosphate buffer vehicle systems, isotonic boric acid vehicles, isotonic sodium chloride vehicles, isotonic sodium borate vehicles, cellulose derivatives, mineral oil, liquid lanolin, white petroleum, sodium citrate, sodium acetate, carbopol, polyvinyl alcohol, polyvinyl pyrrolidone, isopropyl myristate, propylene glycol, polyoxyethylene, polyoxypropylene compound, emulsifying wax, vinylic polymers, polyvinylpyrolidone, polyvinyl alcohol, polyethylene glycol, petrolatum, hyaluronic acid, and talcum.

In some embodiments, the water can be deionized water, or can be a mixture of water and water-miscible solvents.

In some embodiments, the water-miscible solvents can be a lower alkanol or aralkanol, a vegetable oil, polyalkylene glycol, petroleum based jelly, ethyl cellulose, ethyl oleate, carboxymethyl-cellulose, polyvinylpyrrolidone, isopropyl myristate, a solution of medium chain triglycerides (for example, fractionated cocoanut oil), a fixed oil, or combinations thereof.

In some embodiments, the cellulose derivative can be a cellulosic polymer, methylcellulose, sodium carboxymethyl cellulose, hydroxyethyl cellulose, hydroxypropyl cellulose, and hydroxypropylmethyl cellulose.

In some embodiments, the subject is a vertebrate selected from the group consisting of amphibians, reptiles, birds, and mammals.

In some embodiments, a pharmaceutical formulation adapted for topical administration comprising an N-substituted-14-hydroxydihydronormorphine and one or more additives is prepared. In some embodiments, the N-substituted-14-hydroxydihydronormorphine is nalbuphine.

In some embodiments, a method of treating eye pain comprising local administration of an N-substituted-14-hydroxydihydronormorphine of Formula (I) to a subject in need thereof is disclosed. In some embodiments, the N-substituted-14-hydroxydihydronormorphine is nalbuphine.

In some embodiments a pharmaceutical formulation adapted for topical administration, comprising an N-substituted-14-hydroxydihydronormorphine and one or more additives is disclosed.

Accordingly, it is an object of the presently disclosed subject matter to provide novel methods of and compositions for treating pain in a subject in need thereof. This and other objects are achieved in whole or in part by the presently disclosed subject matter.

### DETAILED DESCRIPTION

Disclosed herein is the use of a formulation comprising an N-substituted-14-hydroxydihydronormorphine as a local anesthetic, and in some embodiments a local topical anesthetic. An example of an N-substituted-14-hydroxydihydronormorphine is nalbuphine.

Nalbuphine can be administered topically without a DEA number, enabling nalbuphine to provide needed relief to a subject without pharmaceutical registration, permit, or processing delays. The advantages of prescribing nalbuphine by telephone, allowing prescription refills, and reducing burdensome record keeping are of benefit to both the patient and health care provider.

In addition, nalbuphine exhibits desirable storage capabilities. For example, unlike morphine solutions, refrigeration and protection from light are not required for nalbuphine solutions. A side effect observed in connection with nalbuphine is drowsiness attributed to pain relief, which serves to facilitate relaxation in the patient. Further, the side effects associated with nalbuphine, unlike morphine, do not escalate as dosage is increased.

I. Definitions

It is to be understood that the presently disclosed subject matter is not limited to specific formulations, i.e., specific carrier materials or the like, to specific dosage regimens or to specific drug delivery systems, as such can vary. It is also to be understood that the terminology used herein is for the purpose of describing particular embodiments only and is not intended to be limiting.

Unless defined otherwise, all technical and scientific terms used herein have the same meaning as commonly understood to one of ordinary skill in the art to which the presently disclosed subject matter belongs. Although any methods and materials similar or equivalent to those described herein can be used in the practice or testing of the presently disclosed subject matter, representative methods and materials are now described.

Following long-standing patent law convention, the terms “a”, “an”, and “the” refer to “one or more” when used in this application, including the claims. Thus, for example, reference to “a carrier” includes mixtures of two or more carriers, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the terms “effective amount” and “therapeutically effective amount” are used interchangeably and mean a dosage sufficient to provide treatment for the disease state being treated. This can vary depending on the patient, the disease, and the treatment being effected.

The term “topical administration” is used in its conventional sense to mean delivery of a drug or pharmacologically active agent to the skin or mucosa. For example, the treatment of the eye can be accomplished by topical administration.

The terms “drug” and “pharmacologically active agent” are used herein interchangeably to refer to a chemical material or compound that can induce a desired biological effect in a subject.

“Pharmaceutically acceptable carrier” as used herein includes any and all additives which are acceptable in the pharmaceutical sciences, and can include, for example, high molecular weight polymeric agents such as a cellulosic polymer, hydroxyethylcellulose, hydroxypropylcellulose, methylcellulose, a vinylic polymer, polyvinylpyrolidone, polyvinyl alcohol, polyethylene glycol, petrolatum, talcum or other additives or binders.

The term “topical vehicle” or “topical carrier” as used herein refers to a vehicle suitable for topical application of a drug, and includes any such materials known in the art, e.g., any liquid or nonliquid carrier, gel, cream, ointment, lotion, paste, emulsifier, solvent, liquid diluent, or the like, which is stable with respect to one or more components (in some embodiments all components) of the topical pharmaceutical formulation.

The term “local anesthetic” as used herein refers to a composition that acts via loss of sensation in a localized area of administration in the body.

When the term “N-substituted-14-hydroxydihydronormorphine” is used herein, it is to be understood that the free compound, prodrug, or any pharmaceutically acceptable salts thereof which have anesthetic properties in humans and other vertebrates are included by the term. Such salts include the hydrochloride, hydrobromide, hydroiodide, sulfate, bisulfate, nitrate, citrate, tartrate, bitartrate, phosphate, malate, maleate, fumarate, succinate, acetate and pamoate. It is to be further understood that prodrugs are further included by the term, and that related compounds such as those disclosed in U.S. Pat. No. 3,393,197 are further included by the term. Further, the term “N-substituted-14-hydroxydihydronormorphine” particularly includes nalbuphine, but is not limited exclusively to nalbuphine.

The term “drop” as used herein refers to an amount corresponding to one minim or approximately 1/20 ml.

A subject treated in the embodiments disclosed herein is desirably a vertebrate, and it is to be understood that the principles of the presently disclosed subject matter indicate that the subject matter is effective with respect to all vertebrate species, which are intended to be included in the terms “subject” and “patient”. In this context, a vertebrate is understood to include includes any amphibian, reptile, bird, and mammalian species in which treatment is desirable, particularly agricultural and domestic mammalian species such as humans, horses, cows, pigs, dogs, and cats. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economical importance, such as animals raised on farms for consumption by humans; and/or animals of social importance to humans, such as animals kept as pets or in zoos. Examples of such animals include but are not limited to: primates, including humans, apes and monkeys; carnivores such as cats, dogs, ferrets, wolves, foxes, and coyotes; swine, including pigs, hogs, and wild boars; rodents, such as guinea pigs, hamsters, gerbils, mice, squirrels and beavers; ruminants and/or ungulates such as cattle, antilope, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, doves, chickens, ducks, pigeon, pheasant, geese, guinea fowl, and the like, as they are also of economical importance to humans. Thus, also provided is the treatment of livestock, including, but not limited to, domesticated swine, ruminants, ungulates, horses (including race horses), poultry, Z-Doc, zonk, and the like. Further provided is the treatment of amphibians and reptiles, including the treatment of those species that are endangered and/or kept in zoos. Therefore, also provided is the treatment of amphibians and reptiles, including but not limited to turtles, snakes, lizards, alligators, crocodiles, frogs and toads.

II. General Considerations

Representative N-substituted-14-hydroxydihydronormorphines and their preparation are described in U.S. Pat. No. 3,393,197, the disclosure of which is hereby incorporated by reference. Upon a review of the present disclosure synthesis routes for other such compounds would be apparent to one of ordinary skill in the art.

In accordance with the presently disclosed subject matter, a representative structure of the N-substituted-14-hydroxydihydronormorphines is disclosed in Formula (I):

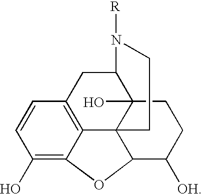

In some embodiments, the R group of Formula (I) is alkyl, substituted alkyl, cycloalkyl, substituted cycloalkyl, aryl, or substituted aryl. In some embodiments, the R group of Formula (I) is selected from the group consisting of allyl, methylallyl, dimethylallyl, propynyl, methylpropynyl, cyclopropylmethyl, cyclobutylmethyl, and propyl.

In some embodiments, the compound of Formula (I) is nalbuphine.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, methylpropynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Further, as used herein, the terms alkyl and/or “substituted alkyl” include an “allyl” or an “allylic group.” The terms “allylic group” or “allyl” refer to the group —CH₂HC═CH₂ and derivatives thereof formed by substitution. Thus, the terms alkyl and/or substituted alkyl include allyl groups, such as but not limited to, allyl, methylallyl, di-methylallyl, and the like. The term “allylic position” or “allylic site” refers to the saturated carbon atom of an allylic group. Thus, a group, such as a hydroxyl group or other substituent group, attached at an allylic site can be referred to as “allylic.”

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, allyloxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

A structure represented generally by a formula such as: 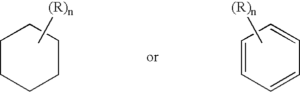
as used herein refers to a ring structure, for example, but not limited to a 3-carbon, a 4-carbon, a 5-carbon, a 6-carbon, and the like, aliphatic and/or aromatic cyclic compound comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure: 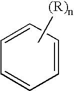
wherein n is an integer from 0 to 2 comprises compound groups including, but not limited to: 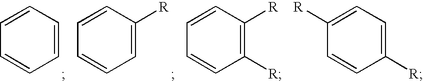
and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)q—N(R)—(CH₂)ᵣ—, wherein each of q and r is independently an integer from 0 to about 20, e.g., 0, 1, 2, 3, 4, 5, 6, 7, 8, 9, 10, 11,12,13,14,15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinabove, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

The term “amino” refers to the —NH₂ group.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

When the term “independently selected” is used, the substituents being referred to (e.g., R groups, such as groups R₁ and R₂, or groups X and Y), can be identical or different. For example, both R₁ and R₂ can be substituted alkyls, or R₁ can be hydrogen and R₂ can be a substituted alkyl, and the like.

Compared with morphine, nalbuphine drastically reduces induction of addiction and accretion of dosage in patients. In addition, nalbuphine has shown decreased inhibition compared to morphine on the respiratory system of the subject treated. Further, it is believed in the art that the depressant effects of nalbuphine on the respiratory system does not increase proportionately with increasing doses, which makes the drug safer in patients at risk from respiratory depression.

Up until now, the use of N-substituted-14-hydroxydihydronormorphines has been limited to oral or parenteral administration, such as capsules, tablets, and powers, or in liquid dosages, such as elixirs, syrups, and suspensions. Disclosed herein for the first time are preparations of N-substituted-14-hydroxydihydronormorphines useful in local administration, and in some embodiments, topical local administration.

III. Formulations

The anesthetic formulations disclosed herein comprise an N-substituted-14-hydroxydihydronormorphine (e.g., nalbuphine). In some embodiments, the anesthetic formulations consist essentially of an N-substituted-14-hydroxydihydronormorphine (e.g., nalbuphine).

Further, in some embodiments the formulations can comprise one or more additional components, also referred to herein as additives, which can be present in ophthalmic or other topical formulations. For example, buffers, wetting agents, stabilizers, tonicity agents and the like can be present in the formulations, which aid in making the anesthetic compositions more comfortable to the user. Further, the anesthetic formulations can be sterilized and can be mixed with auxiliary agents, e.g., surfactants, lubricants, diluents, preservatives, stabilizers, antibacterial agents, solubilizers, surface modifiers, and/or carriers and the like. Such additional components or other additional formulation components can be selected as needed based on particular applications.

The anesthetic formulations can further comprise in some embodiments one or more buffers including, but not limited to, sodium phosphate, sodium phosphate monobasic, sodium acetate, sodium dihydrogen phosphate, sodium hydrogen phosphate, potassium dihydrogen phosphate, potassium hydrogen phosphate, boric acid, sodium borate, citric acid, tartaric acid, sodium tartrate, borate, citrate, bicarbonate, TRIS and various mixed phosphate buffers (including combinations of Na₂HPO₄, NaH₂PO₄ and KH₂PO₄) and mixtures thereof or other agents known to those skilled in the art. Generally, buffers are used in amounts ranging from about 0.3% to about 2% by weight, and any value there between, including but not limited to 0.5,1.0, and 1.5% by weight.

The anesthetic formulations can comprise one or more wetting agent. Such compounds can enhance the viscosity of the composition, and include, but are not limited to: Tween 80, monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”); dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, and carbomer 974P. Such wetting agents can range from about 0.01% to about 5% weight/volume, and any value there between, including but not limited to 0.1, 0.5, 1.0, 1.5, 2.0, 2.5, 3.0, 3.5, 4.0, and 4.5% weight/volume.

The anesthetic formulations can further comprise one or more stabilizers, including but not limited to sodium citrate, disodium edentate, polyvinyl alcohol, hydroxypropylmethylcellulose, glycerin, EDTA, EDTA sodium, carboxymethylcellulose, hydroxyethylcellulose, camphor, chlorobutanol, or other agents known to those skilled in the art. The stabilizers can be present in the range of about 0.01% to about 10% by weight, and any value there between, including but not limited to 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, and 9.5% by weight.

The formulations of the presently disclosed subject matter can be adjusted with tonicity agents to approximate the tonicity of normal bodily fluids. As one example, for ophthalmic use, the formulations of the presently disclosed subject matter can approximate the tonicity of lacrimal fluids, equivalent to a 0.9% solution of sodium chloride or 2.8% of glycerol solution. The formulations can be made substantially isotonic with physiological saline used alone or in combination with other tonicity agents. Correspondingly, excess salt or other tonicity agents can result in the formation of a hypertonic solution that will cause stinging and/or eye irritation. Useful tonicity agents include, but are not limited to, sodium chloride, potassium chloride, mannitol, dextrose, glycerin, propylene glycol and mixtures thereof. In some embodiments, an osmolality of about 225 to about 400 mOsm/kg can be established, including any value there between, and in some embodiments an osmolality of about 280 to about 320 mOsm/kg can be established, including any value there between.

A formulation of the presently disclosed subject matter can contain a thickener, such as, hydroxymethylcellulose, hydroxyethylcellulose, hydroxypropylmethylcellulose, methylcellulose, polyvinyl-pyrrolidone, polysorbate 80, polysorbate 20, or the like, to improve the retention of the formulation. In some embodiments, the thickeners can be incorporated into the composition in an amount ranging from about 0.01% to about 10% by weight and any value there between, including but not limited to 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, and 9.5% by weight.

The formulations can further comprise a lubricant in some embodiments. Any known, suitable lubricants can be used; for example, cellulose derivatives. When present, the formulation can comprise a lubricant in an amount ranging from about, for example, 0.01 to about 5% by weight, or in some embodiments from about 0.05 to about 2% by weight, and in other embodiments from about 0.1 to about 1% by weight of the composition, and including any value between any of the noted ranges, including but not limited to 0.1, 0.5, 1.0, 1.5, 2.0, 2.5, 3.0, 3.5, 4.0, and 4.5% by weight.

The formulations can further comprise a diluent. Suitable diluents can include pharmaceutically acceptable inert fillers, such as, for example, microcrystalline cellulose, lactose, dibasic calcium phosphate, saccharides, microcrystalline cellulose, lactose such as lactose monohydrate, lactose anhydrous, dibasic calcium phosphate, mannitol, starch, sorbitol, sucrose, and glucose or mixtures thereof. Further, the anesthetic formulations can be carried in an inert, non-eye irritating, non-toxic eye drop diluent of conventional formulation, as would generally be understood by one of skill in the art. Such formulations are known, and referred to in, for example, the Physician's Desk Reference for Ophthalmology (1982 Edition, published by Medical Economics Company, Inc., Oridell, N.J.), wherein numerous sterile ophthalmologic ocular solutions are reported, e.g., see pp. 112-114, which are incorporated herein by reference.

In some embodiments, the anesthetic formulations can comprise one or more preservatives including but not limited to paraben, benzalkonium chloride, thimerosal, chlorobutanol, methyl paraben, propyl paraben, phenylethyl alcohol, edetate disodium, sorbic acid, polyquaternium-1, and other agents known to those skilled in the art. Such preservatives can be employed at any desired level, for example, a level of from about 0.001 to about 1.0% weight/volume (“% w/v”).

Further, in some embodiments, the presently disclosed formulations can comprise one or more anti-microbial agents. “Antimicrobial agents” as used herein refers to compositions that derive their antimicrobial activity through a chemical or physiochemical interaction with target microbial organisms. Suitable antimicrobial agents include, but are not limited to, benzalkonium chloride and chlorobutanol, thimerosal, benzalkonium chloride, methyl and propyl paraben, benzyldodecinium bromide, benzyl alcohol, or phenylethanol. In the embodiments of the anesthetic formulation comprising an antimicrobial agent, the antimicrobial agent can be used in an amount that will at least partially reduce the microorganism population in the formulations employed or on/in a subject where the formulation is administered. In some embodiments, a disinfecting amount is used, which is an amount that reduces the microbial burden by two log orders in four hours and in some embodiments by one log order in one hour. In some embodiments, a disinfecting amount is an amount which will eliminate substantially all of the microbial burden. Typically, such agents are present in concentrations ranging from about 0.00001 to about 0.5% (w/v), and more preferably, from about 0.00003 to about 0.05% (w/v).

The formulations in some embodiments can comprise one or more solubilizers including, but not limited to, polyoxyethylene glycol ethers (e.g. sodium carboxymethylcelullose, polyoxyethylene lauryl ether, polyoxyethylene oleyl ether, etc.), polyethylene glycol higher fatty acid esters (e.g. polyethylene glycol monolaurate, polyethylene glycol monooleate, etc.), polyoxyethylene fatty acid esters (e.g. polyoxyethylene sorbitan monolaurate, polyoxyethylene sorbitan monooleate, etc.) or other agents known to those of ordinary skill in the art.

In some embodiments, the formulations can comprise surface modifiers. Representative non-limiting examples of surface modifiers suitable for inclusion within the presently disclosed subject matter include cetyl pyridinium chloride, gelatin, casein, lecithin (phosphatides), dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters (e.g., the commercially available Tweens, such as e.g., Tween 20 and Tween 80, polyethylene glycols (e.g., Carbowaxs 3350 and 1450, and Carbopol, dodecyl trimethyl ammonium bromide, polyoxyethylenestearates, colloidal silicon dioxide, phosphates, sodium dodecylsulfate, carboxymethylcellulose calcium, hydroxypropyl cellulose (HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetaamethylbutyl)-phenol polymer with ethylene oxide and formaldehyde (also known as tyloxapol, superione, and triton), poloxamers, poloxamnines, a charged phospholipid, dialkylesters of sodium sulfosuccinic acid Tritons X-200, Crodestas F-110, p-isononylphenoxypoly-(glycidol), polyoxyethylene sorbitan monooleate, polyoxyl 40 stearate, polyxamer 188, or other agents known to those of ordinary skill in the art.

The formulations disclosed herein can also comprise a physiologically compatible carrier, as those skilled in the art would appreciate and can select using conventional criteria. The carriers can be selected from ophthalmic vehicles which include, but are not limited to, saline solution, deionized water, or mixtures of water and water-miscible solvents such as, for example, lower alkanols or arylalkanols, phosphate buffer vehicle systems, isotonic vehicles such as boric acid, sodium chloride, sodium citrate, sodium acetate and the like, vegetable oils, polyalkylene glycols, and petroleum based jelly, as well as aqueous solutions containing ethyl cellulose, carboxymethyl cellulose and derivatives thereof, hydroxypropylmethyl cellulose, hydroxyethyl cellulose, carbopol, polyvinyl alcohol, polyvinyl pyrrolidone, carbopol-940, isopropyl myristate and other conventionally-employed non-toxic, pharmaceutically acceptable organic and inorganic carriers water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

The pH of the anesthetic formulations can be maintained at a range suitable for use in a particular subject or region of a subject. For example, topical ophthalmic anesthetic formulations of the presently disclosed subject matter can in some embodiments have a range of from about 4 to about 8.5, more preferably from about 6.5 to about 7.5, most preferably about 7 to about 7.5.

IV. Methods of Use

IV.A. Dosage

The anesthetic formulations can conveniently be presented in unit dosage and can be prepared by methods known to one of skill in the art, by bringing the active compound into association with a carrier or diluent, as a suspension or solution, and optionally one or more accessory ingredients, e.g. buffers, antioxidants, surfactants, stabilizers, and the like, as described herein.

In some embodiments, for example, the formulation comprises a topical ophthalmic formulation and comprises an N-substituted-14-hydroxydihydronormorphine at a concentration of from about 0.1% to about 5% by weight and any value there between, including but not limited to 0.1, 0.5, 1.0, 1.5, 2.0, 2.5, 3.0, 3.5, 4.0, 4.5, and 5.0% by weight. In other embodiments of the topical ophthalmic formulation, the concentration of N-substituted-14-hydroxydihydronormorphine is about 1% by weight.

Anesthetic ophthalmic topical anesthetic formulations, in accordance with the presently disclosed subject matter, can be used by instilling, for example, from about 1 to about 2 drops in the affected eye(s) as needed. For example, a first drop of an N-substituted-14-hydroxydihydronormorphine 1% solution can be administered to a subject before surgery and a second drop can be administered after surgery to effectively treat affected eyes for pain or discomfort. Further, the N-substituted-14-hydroxydihydronormorphine 1% solution can be administered up to six times per day (every four hours for 24 hours).

Compositions comprising anesthetic formulations according to the presently disclosed subject matter can be prepared, for example, in unit dosage form or in a more concentrated form that can be diluted at the point of administration.

The presently disclosed subject matter provides a method of treating pain that comprises topically administering to a subject in need of such treatment from about 0.5 to about 1 mg, in some embodiments from about 2 to about 4 mg, an in other embodiments from about 4 to about 8 mg of an N-substituted-14-hydroxydihydronormorphine. In some embodiments, the salt of an N-substituted-14-hydroxydihydronormorphine can be administered instead or in combination with an N-substituted-14-hydroxydihydronormorphine. If a salt of an N-substituted-14-hydroxydihydronormorphine is administered, the salt is present in an amount that, when administered to a patient, produces in the patient the same concentration of the N-substituted-14-hydroxydihydronormorphine, as would administration of the N-substituted-14-hydroxydihydronormorphine.

The dosage administered will, of course, vary depending upon known factors such as age, health, and weight of the recipient, nature and extent of symptoms, kind of concurrent treatment, frequency of treatment, species of subject, and the effect desired.

IV.B. Administration

The presently disclosed formulations can be administered (in some embodiments topically administered) to treat pain by any approach that produces contact of the active agent with the agent's envisioned site of action in the body of a subject, i.e. local administration. The formulations of the presently disclosed subject matter can be administered by any conventional devices and/or techniques available for use in conjunction with pharmaceuticals. The formulation can be administered including a pharmaceutical carrier selected on the basis of the desired site of administration.

One method of administering the pharmaceutical formulation for ophthalmic applications involves administering a liquid or liquid suspension comprising an N-substituted-14-hydroxydihydronormorphine as disclosed herein in the form of eye drops or eyewash to the subject in need thereof. Liquid pharmaceutical compositions of the active compound for producing the eye drops can be prepared by combining the active compound with a suitable vehicle, such as sterile pyrogen free water or sterile saline by techniques disclosed herein and known to one of ordinary skill in the art.

It is envisioned that the presently disclosed subject matter can be administered as a topical solution. It is further envisioned that the presently disclosed subject matter can be administered as an anhydrous ointment comprising a blend of different molecular weight petrolatums, including but not limited to white petroleum, mineral oil, and light mineral oil. It is envisioned that the presently disclosed subject matter can also be administered as an oil-in-water or water-in-oil emulsion in the form of an ointment, cream or drop. It is further envisioned that the presently disclosed subject matter can be administered as an ocular insert or a subconjunctival injection.

When a prodrug or pharmaceutically acceptable salt of an N-substituted-14-hydroxydihydronormorphine (e.g., nalbuphine) is used as a topical anesthetic as described herein, the amount of the salt or prodrug may not necessarily lie within the ranges stated herein. As already noted, if a salt or prodrug of an N-substituted-14-hydroxydihydronormorphine (e.g., nalbuphine) is administered, the salt or prodrug is present in an amount that, when administered to a patient, produces in the patient the same concentration of the N-substituted-14-hydroxydihydronormorphine (e.g., nalbuphine), as would administration of the N-substituted-14-hydroxydihydronormorphine (e.g., nalbuphine). The term “effective amount” encompasses such amounts of prodrugs or salts.

### EXAMPLES

### Example 1

A topical solution of the presently disclosed subject matter is prepared by combining the indicated amounts of the following ingredients:
1) An N-substituted-14-hydroxydihydronormorphine (e.g., nalbuphine), at 1% by weight;
2) One or more of the following agents, totaling 99% by weight:
   a. Benzalconium EDTA (0.01% to 0.1% by weight)
   b. EDTA Sodium (0.005% to 0.1% by weight)
   c. Potassium Chloride (0.05% to 0.9% by weight)
   d. Carboxy Methylcellulose (0.25% to 6% by weight)
   e. Mannitol (1.5% to 2% by weight; in some embodiments 15% to 20% by weight)
   f. Carbomer 934P (0.5% to 2% by weight)
   g. Boric Acid (0.1% to 1.22% by weight)
   h. Polyvinyl Alcohol (0.25% to 3% by weight)
   i. Povidone (1.2% to 10% by weight)
   j. Sodium Phosphate Monobasic (0.5% to 0.9% by weight)
   k. Polysorbate 20 (0.05% to 3% by weight)
   l. Tyloxapol (0.1% to 2% by weight)

Although a topical solution is herein prepared, it is provided that the presently disclosed subject matter can alternatively be administered as an anhydrous ointment comprising a blend of different molecular weight petrolatums, including but not limited to white petroleum, mineral oil, and light mineral oil. The presently disclosed subject matter can further be administered as an oil-in-water or water-in-oil emulsion in the form of an ointment, cream or drop. It is further envisioned that the presently disclosed subject matter can be administered as an ocular insert or a subconjunctival injection.

### Example 2

A pharmaceutical composition of the presently disclosed subject matter is prepared as follows:
1. Dissolve 2 grams of nalbuphine HCl, 1.1 grams citric acid anhydrous, and 0.94 grams sodium citrate anhydrous into 250 ml preserved water.
2. Bring to a total volume of 5 ml with preserved water.
3. Filter into one or more sterile vials using a 0.22 micron filter.

### Example 3

Studies of the pharmaceutical compositions have been performed as follows:
1. The pharmaceutical composition described in Example 1 or 2 was tested.
2. In each study, solutions of 1% Nalbuphine 5 ml drops were administered to domestic animals in the following manner:
   a. One drop of the Nalbuphine 1% solution is administered into the eye of the subject after surgery.
   b. One drop of the Nalbuphine 1% solution is instilled in the eye of the subject when the subject leaves the facility.
3. Results: Effectively relieves eye discomfort. A canine subject with inoperable malignancy of the left eye is to be treated with Nalbuphine 1% for the remainder of its life expectancy. The Nalbuphine 1% offers reduced frequency of dosing, reduced expense, improved compliance, and less pronounced side effects.

### Example 4

Studies of the effects of topically applied Nalbuphine 1% solutions on corneal sensitivity in normal canines have been performed as follows:
1. The pharmaceutical composition described in Example 1 or 2 was tested.
2. In each study, solutions of 1% Nalbuphine drops were administered to sixteen healthy canines of varying age, breed and weight in the following manner:
   a. One drop of Nalbuphine 1% solution was administered topically to the randomly assigned treatment eye.
   b. Artificial tear solution was administered to the contralateral eye, which served as the control.
   c. The corneal touch threshold was determined in using a Cochet-Bonnet aesthesiometer according to well-known methods.
   d. Readings were taken prior to, 15, and 30 minutes post-treatment.
3. Results:
   a. A statistically significant decrease in corneal sensitivity in the Nalbuphine-treated eye 30 minutes post administration was observed.
   b. None of the dogs showed any signs of local irritation post-drug administration.
4. Conclusions:
   a. Nalbuphine 1% increases corneal touch threshold in the healthy canine.
   b. Nalbuphine 1% is well tolerated topically.
   c. Nalbuphine 1% is a viable alternative to topical ocular analgesia in the canine.

### REFERENCES

The references listed below as well as all references cited in the specification are incorporated herein by reference to the extent that they supplement, explain, provide a background for or teach methodology, techniques, and/or compositions employed herein.

Blumberg et al., Pharmacologist 10, 189, Fall 1968.

de Cazanove, et al., “Determination of Nalbuphine in Human Plasma by High-performance Liquid Chromatography with Electrochemical Detection Application to a Pharmacokinetic Study” J. of Chromatography B (1997) vol. 690, pp. 203-210.

Dube, et al., “Determination of Nalbuphine by High-performance Liquid Chromatography with Electrochemical Detection: Application to Clinical Samples from Post-operative Patients” J. of Chromatography (1988) vol. 427, pp. 113-120.

Elliott et al., J. Med. (Basel), 1, 74-89 (1970).

Gear, et al., “Action of Naloxone on Gender-dependent Analgesic and Antianalgesic Effects of Nalbuphine in Humans” J. of Pain (2000), vol. 1(2), pp.122-127.

Gear, et al., “The Kappa Opioid Nalbuphine Produces Genger- and Dose-dependent Analgesia and Antianalgesia in Patients with Postoperative Pain” Pain (1999) vol. 83, pp. 339-345.

Ho, et al., “Determination of Nalbuphine by High-performance liquid Chromatography with Ultraviolet Detection: Application of Human and Rabbit Pharmacokinetic Studies” J. of Chromatography B (1996) vol. 678, pp. 289-269.

Hussain et al., J. Pharm. Sci. 76: 356 (1987).

Hussain et al., Pharm. Res. 5:113 (1988).

Hussain et al., Pharm. Res. 5: 615 (1998).

Lehmann, K. A., Supportive Care in Cancer, (1997) 5/6 (439-444), XP001086567, FIG. 3+L, tables 2, 3.

Lo, et al., “The Disposition and Bioavailability of Intravenous and Oral Nalbuphine in Healthy Volunteers” J. Clin. Pharmacol. (1987) vol. 27, pp. 866-873.

The Merck Index, 12th edition, S. Budavari, ed., 1091-1093 (1996).

Nicolle, et al., “Rapid and Sensitive High-performance Liquid Chromatographic Assay for Nalbuphine in Plasma” J. of Chromatography B (1995) vol. 663, pp. 111-117.

Pao, et al., “High-performance Liquid Chromatographic Method for the Simultaneous Determination of Nalbuphine and its Prodrug, Sebacoyl Dinalburphine Ester, in Dog Plasma and Application to Pharmacokinetic Studies in Dogs” J. of Chromatography B (2000) vol. 746, pp. 241-247.

Remington et al., (1975) Remington's Pharmaceutical Sciences, 15th ed. Mack Pub. Co., Easton, Pa.

Roberts, Drugs of the Future, 2,613-615 (1977).

Sung, et al., “Delivery of Nalbuphine and its Prodrugs Across Skin by Passive Diffusion and lontophoresis” J. of Controlled Release (2000) vol. 67, pp. 1-8.

Wetzelsberger, et al. “Internally Standardized Method for the Determination of Nalbuphine in Human Plasma by Means of High Performance Liquid Chromatography with Electrochemical CoulOmetric Detection” Arzneim-Forsch/Drug Res. (1988) vol. 38(11), pp. 1768-1771.

U.S. Pat. No. 3,393,197 to Pachter et al.

U.S. Pat. No. 3,755,427 to Adams et al.

U.S. Pat. No. 4,237,140 to Dudzinski .

U.S. Pat. No. 4,282,215 to Dudzinski et al.

U.S. Pat. No. 4,464,378 to Hussain.

U.S. Pat. No. 4,573,995 to Chen et al.

U.S. Pat. No. 4,626,539 to Aungst et al.

U.S. Pat. No. 5,512,578 to Crain et al.

U.S. Pat. No. 5,580,876 to Crain et al.

U.S. Pat. No. 5,750,534 to Yoa-Pu et al.

U.S. Pat. No. 5,767,125 to Crain et al.

U.S. Pat. No. 5,840,731 to Mayer et al.

U.S. Pat. No. 6,096,756 to Crain et al.

U.S. Pat. No. 6,225,321 to Hu et al.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for treating pain in a subject in need thereof, comprising topically administering to a site of pain in the subject a therapeutically effective amount of an anesthetic formulation comprising a compound of Formula (I): 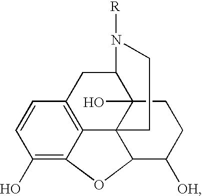
wherein R is selected from the group consisting of alkyl, substituted alkyl, cycloalkyl, substituted cycloalkyl, aryl, and substituted aryl.

2. The method of claim 1, wherein the compound of Formula (l) is nalbuphine.

3. The method of claim 1, wherein the anesthetic formulation is topically administered in the eye of the subject.

4. The method of claim 1, wherein anesthetic formulation comprises an N-substituted-14-hydroxydihydronormorphine at a concentration of about 0.01% to about 5% by weight, optionally at about 1% by weight.

5. The method of claim 1, wherein the anesthetic formulation comprises a pharmaceutically acceptable carrier.

6. The method of claim 1, wherein the anesthetic formulation comprises a pharmaceutically acceptable pH-adjusting agent.

7. The method of claim 6, wherein the anesthetic formulation is adjusted to a pH of from about 6.5 to about 7.

8. The method of claim 1, wherein the anesthetic formulation comprises one or more pharmaceutically acceptable additives selected from the group consisting of a carrier, lubricant, emulsifying agent, wetting agent, bodying agent, tonicity agent, thickener, comfort-enhancing agent, solubilizing aid, antioxidant, stabilizing agent, and combinations thereof.

9. The method of claim 8, wherein the anesthetic formulation comprises one or more opthalmologically acceptable additives selected from the group consisting of a preservative, surfactant, viscosity enhancer, penetration enhancer, buffer, sodium chloride, water and combinations thereof.

10. The method of claim 8, wherein the carrier comprises one or more of water, water-miscible solvents, phosphate buffer vehicle systems, isotonic boric acid vehicles, isotonic sodium chloride vehicles, isotonic sodium borate vehicles, cellulose derivatives, mineral oil, liquid lanolin, white petroleum, sodium citrate, sodium acetate, carbopol, polyvinyl alcohol, polyvinyl pyrrolidone, isopropyl myristate, propylene glycol, polyoxyethylene, polyoxypropylene compound, emulsifying wax, vinylic polymers, polyvinylpyrolidone, polyvinyl alcohol, polyethylene glycol, petrolatum, and talcum.

11. The method of claim 10, wherein the water can be deionized water, or is a mixture of water and water-miscible solvents.

12. The method of claim 10, wherein the water-miscible solvent can be a lower alkanol or aralkanol, a vegetable oil, polyalkylene glycols, petroleum based jelly, ethyl cellulose, ethyl oleate, carboxymethyl-cellulose, polyvinylpyrrolidone, isopropyl myristate, a solution of medium chain triglycerides (for example, cocoanut oil), a fixed oil, or combinations thereof.

13. The method of claim 10, wherein the cellulose derivative can be a cellulosic polymer, methylcellulose, sodium carboxymethyl cellulose, hydroxyethyl cellulose, hydroxypropyl cellulose, and hydroxypropylmethyl cellulose.

14. The method of claim 1, wherein the subject is selected from the group consisting of amphibians, reptiles, birds, and mammals.

15. A method of providing local anesthetic to a subject in need thereof, the method comprising topically administering to a site of pain in the subject an effective amount of an anesthetic formulation comprising a compound of Formula (I): 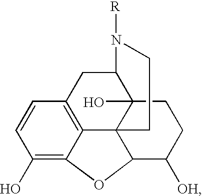
wherein R is selected from the group consisting of alkyl, substituted alkyl, cycloalkyl, substituted cycloalkyl, aryl, and substituted aryl.

16. The method of claim 15, wherein the compound of Formula (I) is nalbuphine.

17. The method of claim 15, wherein the anesthetic formulation is administered as a topical pharmaceutical composition in the eye of the subject.

18. The method of claim 15, wherein anesthetic formulation comprises an N-substituted-14-hydroxydihydronormorphine at a concentration of about 0.01% to about 5% by weight, optionally at about 1% by weight.

19. The method of claim 15, wherein the anesthetic formulation comprises a pharmaceutically acceptable carrier.

20. The method of claim 15, wherein the anesthetic formulation comprises a pharmaceutically acceptable pH-adjusting agent.

21. The method of claim 20, wherein the anesthetic formulation is adjusted to a pH of from about 6.5 to about 7.

22. The method of claim 15, wherein the anesthetic formulation comprises one or more pharmaceutically acceptable additives selected from the group consisting of a carrier, lubricant, emulsifying agent, wetting agent, bodying agent, tonicity agent, thickener, comfort-enhancing agent, solubilizing aid, antioxidant, stabilizing agent, and combinations thereof.

23. The method of claim 22, wherein the anesthetic formulation comprises one or more opthalmologically acceptable composition selected from the group consisting of a preservative, surfactant, viscosity enhancer, penetration enhancer, buffer, sodium chloride, water and combinations thereof.

24. The method of claim 22, wherein the carrier comprises one or more of water, water-miscible solvents, conventional phosphate buffer vehicle systems, isotonic boric acid vehicles, isotonic sodium chloride vehicles, isotonic sodium borate vehicles, cellulose derivatives, mineral oil, liquid lanolin, white petroleum, sodium citrate, sodium acetate, carbopol, polyvinyl alcohol, polyvinyl pyrrolidone, isopropyl myristate, propylene glycol, polyoxyethylene, polyoxypropylene compound, emulsifying wax, vinylic polymers, polyvinylpyrolidone, polyvinyl alcohol, polyethylene glycol, petrolatum, and talcum.

25. The method of claim 24, wherein the water can be deionized water, or is a mixture of water and water-miscible solvents.

26. The method of claim 24, wherein the water-miscible solvent can be a lower alkanol or aralkanol, a vegetable oil, polyalkylene glycol, petroleum based jelly, ethyl cellulose, ethyl oleate, carboxymethyl-cellulose, polyvinylpyrrolidone, isopropyl myristate, a solution of medium chain triglycerides (for example, cocoanut oil), a fixed oil, or combinations thereof.

27. The method of claim 24, wherein the cellulose derivatives can be a cellulosic polymer, methylcellulose, sodium carboxymethyl cellulose, hydroxyethyl cellulose, hydroxypropyl cellulose, and hydroxypropylmethyl cellulose.

28. The method of claim 15, wherein the subject is selected from the group consisting of amphibians, reptiles, birds, and mammals.

29. A method of treating eye pain through the local administration to a subject in need thereof an effective amount of an anesthetic formulation comprising an N-substituted-14-hydroxydihydronormorphine of Formula (I): 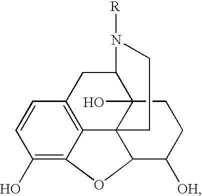
wherein R is selected from the group consisting of alkyl, substituted alkyl, cycloalkyl, substituted cycloalkyl, aryl, and substituted aryl.

30. The method of claim 29, wherein the N-substituted-14-hydroxydihydronormorphine is nalbuphine.

31. A pharmaceutical formulation adapted for topical administration, comprising: comprising an N-substituted-14-hydroxydihydronormorphine of Formula (I): 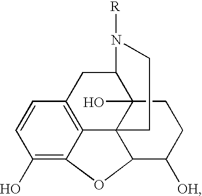
wherein R is selected from the group consisting of alkyl, substituted alkyl, cycloalkyl, substituted cycloalkyl, aryl, and substituted aryl; and one or more additives.

32. The pharmaceutical formulation of claim 31, wherein the N-substituted-14-hydroxydihydronormorphine is nalbuphine.
